(19) United States
(12) Patent Application Publication
(10) Pub. No.: US 2008/0019930 A1
(43) Pub. Date: Jan. 24, 2008

(54) Dibenzoylmethane sunscreens photostabilized with arylalkyl amide or ester compounds

(75) Inventors: Didier Candau, Bievres (FR); Karine Lucet-Levannier, Reuil-Malmaison (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 11/819,037

(22) Filed: Jun. 25, 2007

Related U.S. Application Data
(60) Provisional application No. 60/819,638, filed on Jul. 11, 2006.
(60) Provisional application No. 60/819,637, filed on Jul. 11, 2006.

(30) Foreign Application Priority Data
Jun. 23, 2006 (FR) 0652621
Jun. 23, 2006 (FR) 0652620

(51) Int. Cl. A61K 8/30; A61Q 17/04

(52) U.S. Cl. 424/60; 424/59

(57) ABSTRACT

Improvedly effective and photostable sunscreen compositions combine a dibenzoylmethane screening agent and a specific arylalkyl amide or ester compound of the following formulae (I), (II) or (III):

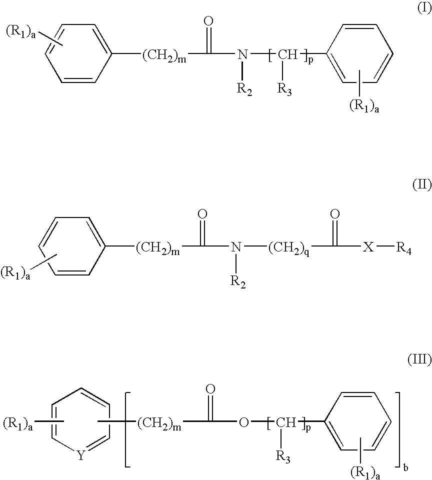

### CROSS-REFERENCE TO PRIORITY/PROVISIONAL APPLICATIONS

This application claims priority under 35 U.S.C. § 119 of FR 0652620, filed Jun. 23, 2006, FR 0652621, filed Jun. 23, 2006, U.S. Provisional Application No. 60/819,637, Jul. 11, 2006 and U.S. Provisional Application No. 60/819,638, Jul. 11, 2006, each hereby expressly incorporated by reference in its entirety and each assigned to the assignee hereof.

### BACKGROUND OF THE INVENTION

1. Technical Field of the Invention

The present invention relates to cosmetic sunscreen compositions comprising the combination of a dibenzoylmethane derivative and of a specific arylalkyl amide or ester compound of formula (I), (II) or (III), as defined below.

This invention also relates to a process for photostabilizing, with regard to radiation, at least one dibenzoylmethane derivative by at least one specific arylalkyl amide or ester compound of formula (I), (II) or (III).

The present invention also relates to the formulation of the said specific arylalkyl amide or ester compound of formula (I), (II) or (III) into a composition comprising, in a cosmetically acceptable carrier, at least one screening agent of the dibenzoylmethane derivative type for improving the effectiveness of the said composition with regard to UV-A rays.

2. Description of Background and/or Related and/or Prior Art

It is known that light radiation with wavelengths of from 280 nm to 400 nm makes possible browning of the human epidermis and that rays with wavelengths more particularly of from 280 to 320 nm, known as UV-B radiation, cause erythemas and skin burns which may be harmful to the development of natural tanning. For these reasons and for aesthetic reasons, there exists a constant demand for means for controlling this natural tanning for the purpose of thus controlling the color of the skin; it is thus advisable to screen out this UV-B radiation.

It is also known that UV-A rays, with wavelengths of from 320 to 400 nm, which cause browning of the skin, are capable of bringing about a detrimental change in the latter, in particular in the case of sensitive skin or of skin continually exposed to solar radiation. UV-A rays bring about in particular a loss of elasticity of the skin and the appearance of wrinkles, resulting in premature cutaneous aging. They promote the triggering of the erythemal reaction or accentuate this reaction in certain individuals and can even be the cause of phototoxic or photoallergic reactions. Thus, for aesthetic and cosmetic reasons, such as the retention of the natural elasticity of the skin, for example, individuals increasingly desire to control the effects of UV-A rays on their skin. It is thus desirable also to screen out UV-A radiation.

With the purpose of providing protection of the skin and keratinous substances against UV radiation, application is generally made of anti-sun or sunscreen compositions comprising organic screening agents which are active in the UV-A region and which are active in the UV-B region. The majority of these screening agents are fat-soluble.

In this regard, a particularly advantageous family of UV-A screening agents is currently composed of dibenzoylmethane derivatives and in particular 4-(tert-butyl)-4′-methoxydibenzoylmethane, this being because these exhibit a high intrinsic absorbency. These dibenzoylmethane derivatives, which are now products well known per se as screening agents active in the UV-A region, are disclosed in particular in FR-A-2,326,405 and FR-A-2,440,933, and also in EP-A-0,114,607; furthermore, 4-(tert-butyl)-4′-methoxydibenzoylmethane is currently marketed under the trademark “Parsol 1789” by DSM Nutritional Products.

Unfortunately, it transpires that dibenzoylmethane derivatives are compounds which are relatively sensitive to ultraviolet radiation (in particular UV-A radiation), that is to say, more specifically, that they exhibit an unfortunate tendency to decompose more or less rapidly under the action of the latter. Thus, this substantial lack of photochemical stability of dibenzoylmethane under the influence of the ultraviolet radiation to which they are by nature intended to be subjected does not make it possible to guarantee constant protection during prolonged exposure to the sun, such that repeated applications at regular and close intervals of time have to be carried out by the user in order to obtain effective protection of the skin against UV rays.

It is known that, in EP-717,982, amide compounds have a photostabilizing effect on dibenzoylmethane derivatives and more particularly amide oils, such as the compound N,N-diethyl-3-methylbenzamide of structure: 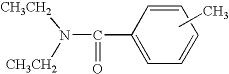
or ethyl N-butyl and N-acetylaminopropionate of formula:

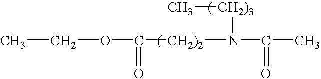

To solve this problem, provision has already been made, in EP-1,618,870, to employ arylalkyl benzoate derivatives, in particular 2-ethylphenyl benzoate, such as the commercial product marketed under ethylphenyl benzoate, such as the commercial product marketed under the trademark X-Tend 226® by ISP. However, the level of photostability of the dibenzoylmethane derivative after exposure to UV radiation still remains inadequate.

### SUMMARY OF THE INVENTION

It has now surprisingly been determined that by combining with the dibenzoylmethane indicated above, an effective amount of a specific arylalkyl amide or ester derivative of formula (I), (II) or (III) as defined below, it is possible to further improve, in a substantial and noteworthy manner, the photochemical stability (or photostability) of these same dibenzoylmethane derivatives and their effectiveness in the UV-A region. The compositions comprising such a combination also result, after application, in a more homogenous distribution of UV screening agent.

Thus, the present invention features compositions comprising, formulated into a cosmetically acceptable carrier, at least one UV screening system which comprises:

(a) at least one UV-A screening agent of the dibenzoylmethane derivative type and

(b) at least one arylalkyl amide or ester compound of formula (I), (II) or (III), as defined below.

This invention also features a process for improving the chemical stability with regard to UV radiation of at least one UV-A screening agent of the dibenzoylmethane derivative type which entails combining, with the said dibenzoylmethane derivative, at least one specific arylalkyl amide or ester compound of formula (I), (II) or (III), as defined below.

Finally, the present invention features the formulation of a specific arylalkyl amide or ester compound of formula (I), (II) or (III) in a composition comprising, in a cosmetically acceptable carrier, at least one dibenzoylmethane derivative for improving the effectiveness of the said composition with regard to UV-A rays.

Other characteristics, aspects and advantages of the invention will become apparent from the detailed description which will follow.

### DETAILED DESCRIPTION OF BEST MODE AND SPECIFIC/PREFERRED EMBODIMENTS OF THE INVENTION

The term “cosmetically acceptable” means compatible with the skin and/or its superficial body growths, which exhibits a pleasant color, a pleasant smell and a pleasant feel, and which does not cause unacceptable discomfort (smarting, tightness, red blotches) liable to dissuade the consumer from using this composition.

Among the dibenzoylmethane derivatives, particularly exemplary are:
2-methyldibenzoylmethane,
4-methyldibenzoylmethane,
4-isopropyldibenzoylmethane,
4-(tert-butyl)dibenzoylmethane,
2,4-dimethyldibenzoylmethane,
2,5-dimethyldibenzoylmethane,
4,4′-diisopropyldibenzoylmethane,
4,4′-dimethoxydibenzoylmethane,
4-(tert-butyl)-4′-methoxydibenzoylmethane,
2-methyl-5-isopropyl-4′-methoxydibenzoylmethane,
2-methyl-5-(tert-butyl)-4′-methoxydibenzoylmethane,
2,4-dimethyl-4′-methoxydibenzoylmethane,
2,6-dimethyl-4-(tert-butyl)-4′-methoxydibenzoylmethane.

Among the dibenzoylmethane derivatives indicated above, preferred is 4-isopropyldibenzoylmethane, marketed under the trademark of “Eusolex 8020” by Merck and corresponding to the following formula:

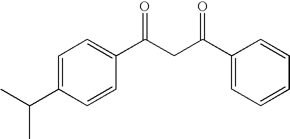

Very particularly preferred is 4-(tert-butyl)-4′-methoxydibenzoylmethane or Butyl Methoxy Dibenzoylmethane, marketed under the trademark of “Parsol 1789” by DSM Nutritional Products; this screening agent corresponds to the following formula:

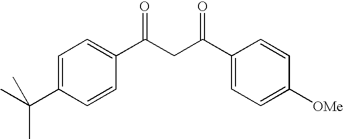

The dibenzoylmethane derivative or derivatives can be present in the compositions in accordance with the invention at contents which preferably vary from 0.01% to 10% by weight and more preferably from 0.1% to 6% by weight, with respect to the total weight of the composition.

The arylalkyl amides or esters in accordance with the invention are selected from among those corresponding to the following general formulae (I), (II) and (III): 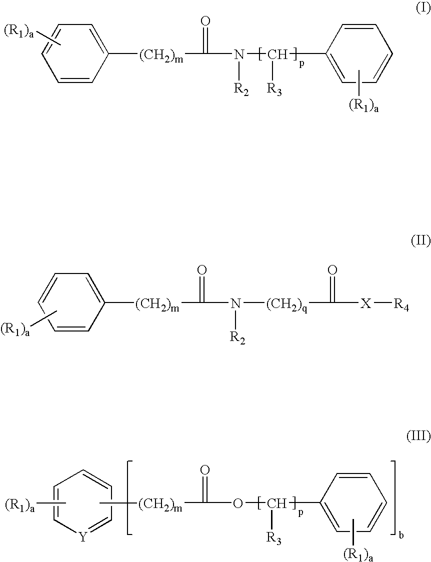
in which:

R₁ is a linear or branched C₁-C₄ alkyl radical,

a is 0-2,

m is 0-4,

b is 1 or 2,

R₂ is H, a linear or branched C₁-C₂₀ alkyl radical or the acetyl group,

R₃ is independently H or methyl,

p is 0-4,

q is 0-3,

X is O, NH or NR₅,

Y is CH or N,

R₄ and R₅ are linear or branched C₁-C₂₀ alkyl radicals, with the proviso that, in formula (III): if Y=CH, R₃=H, m=0, p=2, then a is other than 0 and R₁ is other than methyl.

Particularly exemplary in the formulae (I), (II) and (III), among the alkyl radicals, are the methyl, ethyl, n-propyl, isopropyl, n-butyl, isobutyl, tert-butyl, 2-ethylhexyl, dodecyl or hexadecyl radicals.

Among the amide compounds of formula (I), particularly exemplary are the following compounds (a) to (k):

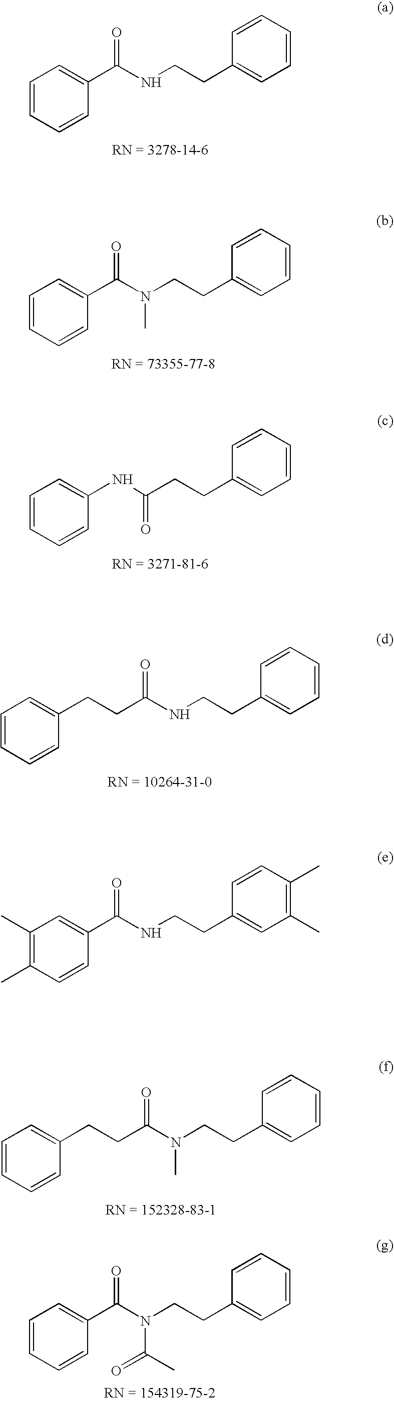

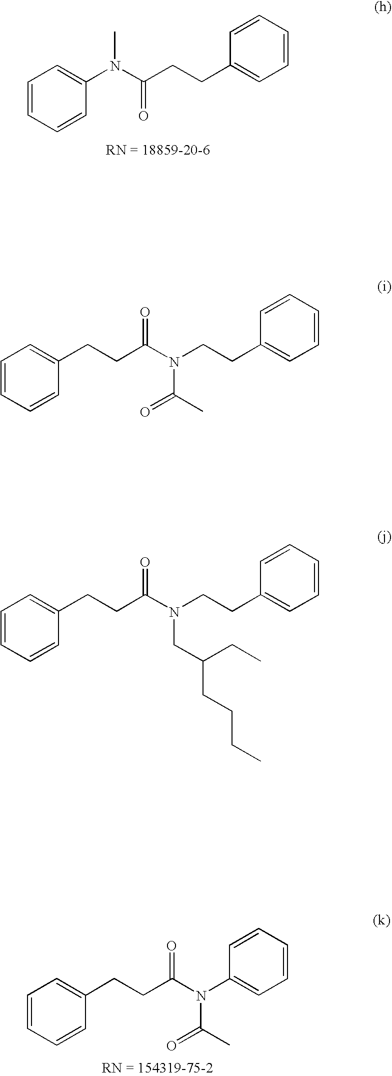

Among the amide compounds of formula (II), preferred are the following compounds (I) to (r):

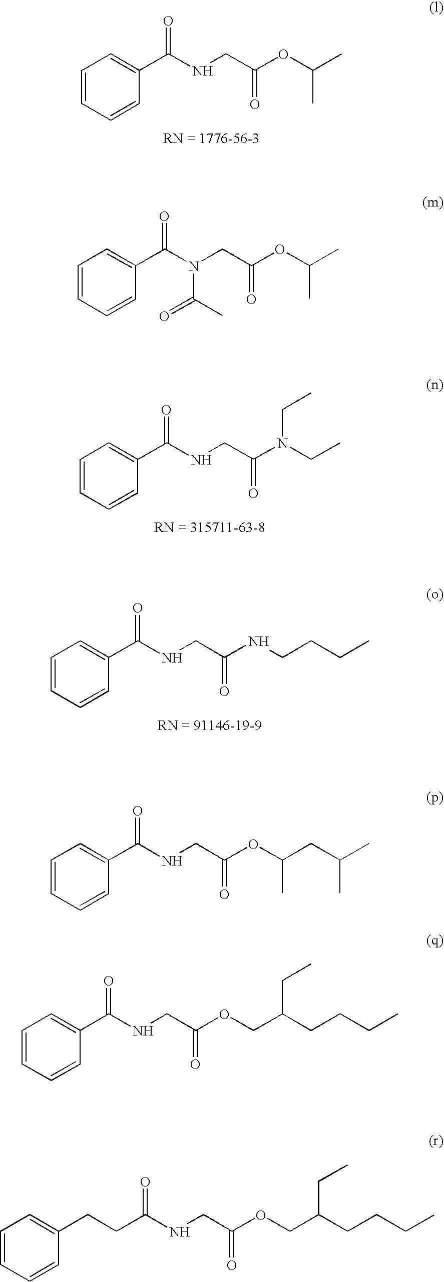

Among the arylalkyl ester compounds of formula (III) indicated above, particularly exemplary are the following compounds (s) to (x):

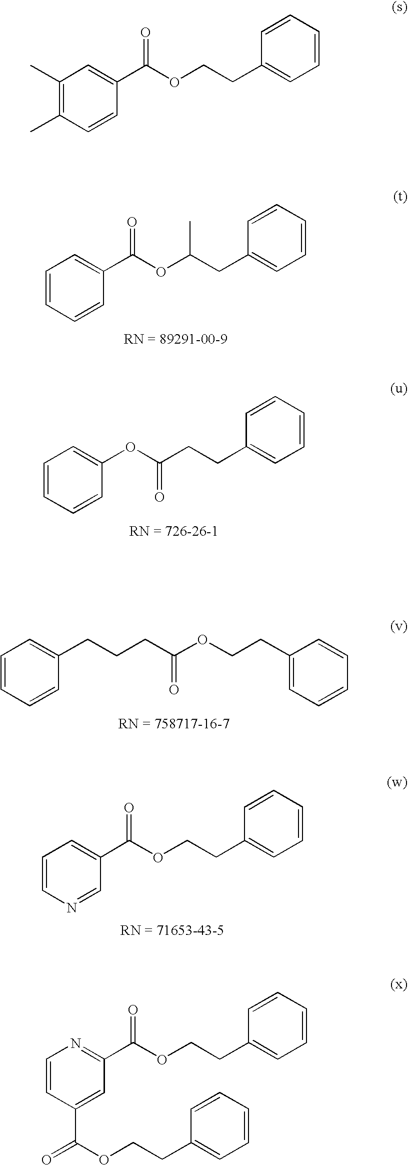

The ester compounds of formula (III) can be prepared by reaction of the acid derivative of formula (2) with the alcohol of formula (3) in the presence of an acid catalyst, such as, for example, concentrated sulfuric acid, in a solvent medium, such as, for example, toluene, according to the following scheme: 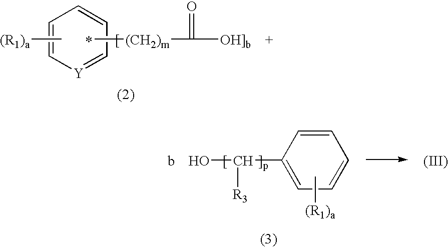
with the radicals R₁, R₂, R₃, a, b, m and p having the same definitions as above.

The amide compounds of formula (I) can be prepared by reaction of the acid chloride of formula (3) with the ester amine of formula (4) in the presence of an HCl scavenger, such as, for example, triethylamine, in a solvent medium, such as, for example, acetonitrile, according to the following scheme: 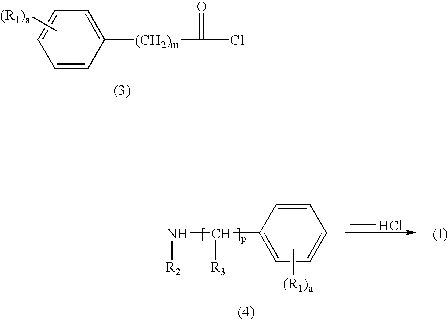
with the radicals R₁, R₂, R₃, a, m and p having the same definitions as above.

The amide compounds of formula (II) can be prepared by reaction of the acid derivative of formula (5) with the alcohol of formula (6) in the presence of an acid catalyst, such as, for example, concentrated sulfuric acid, in a solvent medium, such as, for example, toluene, according to the following scheme:

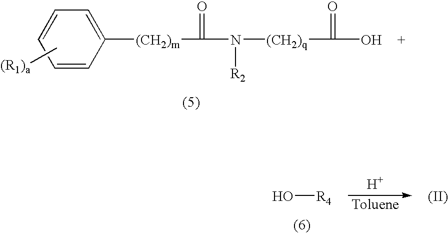

The arylalkyl amide or ester compound or compounds can be present in the compositions at contents which are generally from 0.01% to 20% by weight, and preferably at contents of from 0.1% to 10% by weight, with respect to the total weight of the composition.

According to the present invention, the compound or compounds of formulae (I), (II) and (III) will be used in a sufficient amount which makes it possible to obtain a notable and significant improvement in the photostability of the dibenzoylmethane derivative in a given composition. This minimum amount of photostabilizing agent to be employed can vary according to the amount of dibenzoylmethane present at the start in the composition and according to the nature of the cosmetically acceptable carrier selected for the composition. It can be determined without any difficulty using a conventional test for measuring photostability.

Furthermore, the compositions in accordance with the invention can comprise other additional organic or inorganic UV screening agents active in the UV-A and/or UV-B region which are water-soluble or fat-soluble or else insoluble in the cosmetic solvents commonly used.

The additional organic UV screening agents are selected in particular from among anthranilates; cinnamic derivatives; salicylic derivatives; camphor derivatives; benzophenone derivatives; β,β-diphenylacrylate derivatives; triazine derivatives; benzotriazole derivatives; benzalmalonate derivatives, in particular those described in U.S. Pat. No. 5,624,663; benzimidazole derivatives; imidazolines; bis-benzoazolyl derivatives, such as described in EP-669,323 and U.S. Pat. No. 2,463,264; p-aminobenzoic acid (PABA) derivatives; methylenebis(hydroxyphenylbenzotriazole) derivatives, such as described U.S. Pat. Nos. 5,237,071, 5,166,355, GB-2,303,549, DE-197,26,184 and EP-893,119; benzoxazole derivatives, such as described in EP-0,832,642, EP-1,027,883, EP-1,300,137 and DE-10162844; screening polymers and screening silicones, such as those described in particular in WO 93/04665; dimers derived from α-alkylstyrene, such as those described in DE-19855649; 4,4-diarylbutadienes, such as described in EP-0,967,200, DE-19746654, DE-19755649, EP-A-1,008,586, EP-1,133,980 and EP-133,981; and their mixtures.

Exemplary additional organic UV screening agents are those denoted below under their INCI names:

para-Aminobenzoic Acid Derivatives:

PABA,

Ethyl PABA,

Ethyl Dihydroxypropyl PABA,

Ethylhexyl Dimethyl PABA, marketed in particular under the trademark “Escalol 507” by ISP,

Glyceryl PABA,

PEG-25 PABA, marketed under the trademark “Uvinul P25” by BASF,

Salicylic Derivatives:

Homosalate, marketed under the trademark “Eusolex HMS” by Rona/EM Industries,

Ethylhexyl Salicylate, marketed under the trademark “Neo Heliopan OS” by Haarmann and Reimer,

Dipropyleneglycol Salicylate, marketed under the trademark “Dipsal” by Scher,

TEA Salicylate, marketed under the trademark “Neo Heliopan TS” by Haarmann and Reimer,

Cinnamic Derivatives:

Ethylhexyl Methoxycinnamate, marketed in particular under the trademark “Parsol MCX” by Hoffmann-LaRoche,

Isopropyl Methoxycinnamate,

Isoamyl Methoxycinnamate, marketed under the trademark “Neo Heliopan E 1000” by Haarmann and Reimer,

Cinoxate,

DEA Methoxycinnamate,

Diisopropyl Methylcinnamate,

Glyceryl Ethylhexanoate Dimethoxycinnamate,

β,β-Diphenylacrylate Derivatives:

Octocrylene, marketed in particular under the trademark “Uvinul N539” by BASF,

Etocrylene, marketed in particular under the trademark “Uvinul N35” by BASF,

Benzophenone Derivatives:

Benzophenone-1, marketed under the trademark “Uvinul 400” by BASF,

Benzophenone-2, marketed under the trademark “Uvinul D50” by BASF,

Benzophenone-3 or Oxybenzone, marketed under the trademark “Uvinul M40” by BASF,

Benzophenone-4, marketed under the trademark “Uvinul MS40” by BASF,

Benzophenone-5,

Benzophenone-6, marketed under the trademark “Helisorb 11” by Norquay,

Benzophenone-8, marketed under the trademark “Spectra-Sorb UV-24” by American Cyanamid,

Benzophenone-9, marketed under the trademark “Uvinul DS-49” by BASF,

Benzophenone-12,

n-Hexyl 2-(4-diethylamino-2-hydroxybenzoyl)benzoate,

Benzylidenecamphor Derivatives:

3-Benzylidene camphor, manufactured under the trademark “Mexoryl SD” by Chimex,

4-Methylbenzylidene camphor, marketed under the trademark “Eusolex 6300” by Merck,

Benzylidene Camphor Sulfonic Acid, manufactured under the trademark “Mexoryl SL” by Chimex,

Camphor Benzalkonium Methosulfate, manufactured under the trademark “Mexoryl SO” by Chimex,

Terephthalylidene Dicamphor Sulfonic Acid, manufactured under the trademark “Mexoryl SX” by Chimex,

Polyacrylamidomethyl Benzylidene Camphor, manufactured under the trademark “Mexoryl SW” by Chimex,

Phenylbenzimidazole Derivatives:

Phenylbenzimidazole Sulfonic Acid, marketed in particular under the trademark “Eusolex 232” by Merck,

Disodium Phenyl Dibenzimidazole Tetrasulfonate, marketed under the trademark “Neo Heliopan AP” by Haarmann and Reimer,

Phenylbenzotriazole Derivatives:

Drometrizole Trisiloxane, marketed under the trademark “Silatrizole” by Rhodia Chimie,

Methylene Bis-Benzotriazolyl Tetramethylbutylphenol, marketed in the solid form under the trademark “Mixxim BB/100” by Fairmount Chemical or in the micronized form in aqueous dispersion under the trademark “Tinosorb M” by Ciba Specialty Chemicals,

Triazine Derivatives:

Bis-Ethylhexyloxyphenol Methoxyphenyl Triazine, marketed under the trademark “Tinosorb S” by Ciba-Geigy,

Ethylhexyl Triazone, marketed in particular under the trademark “Uvinul T150” by BASF,

Diethylhexyl Butamido Triazone, marketed under the trademark “Uvasorb HEB” by Sigma 3V,

2,4,6-Tris(dineopentyl 4′-aminobenzalmalonate)-s-triazine,

2,4,6-Tris(diisobutyl 4′-aminobenzalmalonate)-s-triazine,

The symmetrical triazine screening agents described in U.S. Pat. No. 6,225,467, WO 2004/085412 (see compounds 6 and 9) or the document “Symmetrical Triazine Derivatives”, IP.COM Journal, IP.COM INC, WEST HENRIETTA, N.Y., US (20 Sep. 2004), in particular the 2,4,6-tris(biphenyl)-1,3,5-triazines (especially 2,4,6-tris(biphenyl-4-yl)-1,3,5-triazine) and 2,4,6-tris(terphenyl)-1,3,5-triazine, which are taken up again in WO 06/035000, WO 06/034982, WO 06/034991, WO 06/035007, WO 2006/034992 and WO 2006/034985.

Anthranilic Derivatives:

Menthyl anthranilate, marketed under the trademark “Neo Heliopan MA” by Haarmann and Reimer,

Imidazoline Derivatives:

Ethylhexyl Dimethoxybenzylidene Dioxoimidazoline Propionate,

Benzalmalonate Derivatives:

Dineopentyl 4′-methoxybenzalmalonate,

Polyorganosiloxane comprising benzalmalonate functional groups, such as Polysilicone-15, marketed under the trademark “Parsol SLX” by Hoffmann-LaRoche,

4,4-Diarylbutadiene Derivatives:

1,1-Dicarboxy(2,2′-dimethylpropyl)-4,4-diphenylbutadiene,

Benzoxazole Derivatives:

2,4-bis[5-1 (dimethylpropyl)benzoxazol-2-yl-(4-phenyl)imino]-6-(2-ethylhexyl)imino-1,3,5-triazine, marketed under the trademark of Uvasorb K2A by Sigma 3V

and their mixtures.

The preferred additional organic photoprotective agents are selected from among:
Ethylhexyl Methoxycinnamate,
Homosalate,
Ethylhexyl Salicylate,
Octocrylene,
Phenylbenzimidazole Sulfonic Acid,
Benzophenone-3,
Benzophenone-4,
Benzophenone-5,
n-Hexyl 2-(4-diethylamino-2-hydroxybenzoyl)benzoate,
4-Methylbenzylidene Camphor,
Terephthalylidene Dicamphor Sulfonic Acid,
Disodium Phenyl Dibenzimidazole Tetrasulfonate,
Methylene Bis-Benzotriazolyl Tetramethylbutylphenol,
Ethylhexyl triazone,
Bis-Ethylhexyloxyphenol Methoxyphenyl Triazine,
Diethylhexyl Butamido Triazone,
2,4,6-Tris(dineopentyl 4′-aminobenzalmalonate)-s-triazine,
2,4,6-Tris-(diisobutyl 4′-aminobenzalmalonate)-s-triazine,
2,4,6-Tris(biphenyl-4-yl)-1,3,5-triazine,
2,4,6-Tris(terphenyl)-1,3,5-triazine,
Drometrizole Trisiloxane,
Polysilicone-15,
Dineopentyl 4′-methoxybenzalmalonate,
1,1-Dicarboxy(2,2′-dimethylpropyl)-4,4-diphenylbutadiene,
2,4-Bis[5-1(dimethylpropyl)benzoxazol-2-yl-(4-phenyl)imino]-6-(2-ethylhexyl)imino-1,3,5-triazine,
   and their mixtures.

The inorganic UV screening agents are selected from among pigments formed of metal oxides which may or may not be coated, having a mean size of the primary particles: generally from 5 nm to 100 nm, preferably from 10 nm to 50 nm, such as, for example, pigments formed of titanium oxide (amorphous or crystalline in the rutile and/or anatase form), iron oxide, zinc oxide, zirconium oxide or cerium oxide, which are all UV photoprotective agents well known per se.

The pigments may or may not be coated.

The coated pigments are pigments which have been subjected to one or more surface treatments of chemical, electronic, mechanochemical and/or mechanical nature with compounds such as described, for example, in Cosmetics & Toiletries, February 1990, Vol. 105, pp. 53-64, such as amino acids, beeswax, fatty acids, fatty alcohols, anionic surfactants, lecithins, sodium, potassium, zinc, iron or aluminum salts of fatty acids, metal alkoxides (titanium or aluminum alkoxides), polyethylene, silicones, proteins (collagen, elastin), alkanolamines, silicon oxides, metal oxides or sodium hexametaphosphate.

In known manner, the silicones are organosilicon polymers or oligomers comprising a linear or cyclic and branched or crosslinked structure, of variable molecular weight, obtained by polymerization and/or polycondensation of suitable functional silanes and essentially composed of a repetition of main units in which the silicon atoms are connected to one another via oxygen atoms (siloxane bond), optionally substituted hydrocarbon radicals being connected directly to the said silicon atoms via a carbon atom.

The term “silicones” also encompasses the silanes necessary for their preparation, in particular alkylsilanes.

The silicones used for the coating of the pigments suitable for the present invention are preferably selected from the group consisting of alkylsilanes, polydialkylsiloxanes and polyalkylhydrosiloxanes. More preferably still, the silicones are selected from the group consisting of octyltrimethylsilane, polydimethylsiloxanes and polymethylhydrosiloxanes.

Of course, the pigments formed of metal oxides may, before their treatment with silicones, have been treated with other surfacing agents, in particular with cerium oxide, alumina, silica, aluminum compounds, silicon compounds or their mixtures.

The coated pigments are more particularly titanium oxides coated:

with silica, such as the product “Sunveil” from Ikeda and the product “Eusolex T-AVO” from Merck

with silica and with iron oxide, such as the product “Sunveil F” from Ikeda,

with silica and with alumina, such as the products “Microtitanium Dioxide MT 500 SA” and “Microtitanium Dioxide MT 100 SA” from Tayca, “Tioveil” from Tioxide and “Mirasun TiW 60” from Rhodia,

with alumina, such as the products “Tipaque TTO-55 (B)” and “Tipaque TTO-55 (A)” from Ishihara and “UVT 14/4” from Kemira,

with alumina and with aluminum stearate, such as the product “Microtitanium Dioxide MT 100 TV, MT 100 TX, MT 100 Z or MT-01 from Tayca and the products “Solaveil CT-10 W”, “Solaveil CT 100” and “Solaveil CT 200” from Uniqema,

with silica, with alumina and with alginic acid, such as the product “MT-100 AQ” from Tayca,

with alumina and with aluminum laurate, such as the product “Microtitanium Dioxide MT 100 S” from Tayca,

with iron oxide and with iron stearate, such as the product “Microtitanium Dioxide MT 100 F” from Tayca,

with zinc oxide and with zinc stearate, such as the product “BR351” from Tayca,

with silica and with alumina and treated with a silicone, such as the products “Microtitanium Dioxide MT 600 SAS”, “Microtitanium Dioxide MT 500 SAS” or “Microtitanium Dioxide MT 100 SAS” from Tayca,

with silica, with alumina and with aluminum stearate and treated with a silicone, such as the product “STT-30-DS” from Titan Kogyo,

with silica and treated with a silicone, such as the product “UV-Titan X 195” from Kemira or the product SMT-100 WRS from Tayca,

with alumina and treated with a silicone, such as the products “Tipaque TTO-55 (S)” from Ishihara or “UV Titan M 262” from Kemira,

with triethanolamine, such as the product “STT-65-S” from Titan Kogyo,

with stearic acid, such as the product “Tipaque TTO-55 (C)” from Ishihara,

with sodium hexametaphosphate, such as the product “Microtitanium Dioxide MT 150 W” from Tayca.

Other titanium oxide pigments treated with a silicone are preferably TiO₂ treated with octyltrimethylsilane and for which the mean size of the individual particles is from 25 and 40 nm, such as that marketed under the trademark “T 805” by Degussa Silices, TiO₂ treated with a polydimethylsiloxane and for which the mean size of the individual particles is 21 nm, such as that marketed under the trademark “70250 Cardre UF TiO2SI3” by Cardre, anatase/rutile TiO₂ treated with a polydimethylhydrosiloxane and for which the mean size of the individual particles is 25 nm, such as that marketed under the trademark “Microtitanium Dioxyde USP Grade Hydrophobic” by Color Techniques.

The uncoated titanium oxide pigments are, for example, marketed by Tayca under the trademarks “Microtitanium Dioxide MT 500 B” or “Microtitanium Dioxide MT600 B”, by Degussa under the trademark “P 25”, by Wacker under the trademark “Oxyde de titane transparent PW”, by Miyoshi Kasei under the trademark “UFTR”, by Tomen under the trademark “ITS” and by Tioxide under the trademark “Tioveil AQ”.

The uncoated zinc oxide pigments are, for example:

those marketed under the trademark “Z-cote” by Sunsmart;

those marketed under the trademark “Nanox” by Elementis;

those marketed under the trademark “Nanogard WCD 2025” by Nanophase Technologies.

The coated zinc oxide pigments are, for example:

those marketed under the trademark “Z-cote HP1” by Sunsmart (ZnO coated with dimethicone);

those marketed under the trademark “Oxide zinc CS-5” by Toshibi (ZnO coated with polymethylhydrosiloxane);

those marketed under the trademark “Nanogard Zinc Oxide FN” by Nanophase Technologies (as a 40% dispersion in Finsolv TN, C₁₂-C₁₅ alkyl benzoate);

those marketed under the trademark “Daitopersion Zn-30” and “Daitopersion Zn-50” by Daito (dispersions in oxyethylenated polydimethylsiloxane/cyclopolymethylsiloxane comprising 30% or 50% of zinc nanooxides coated with silica and polymethylhydrosiloxane);

those marketed under the trademark “NFD Ultrafine ZnO” by Daikin (ZnO coated with phosphate of perfluoroalkyl and copolymer based on perfluoroalkylethyl as a dispersion in cyclopentasiloxane);

those marketed under the trademark “SPD-Z1” by Shin-Etsu (ZnO coated with silicone-grafted acrylic polymer dispersed in cyclodimethylsiloxane);

those marketed under the trademark “Escalol Z100” by ISP (alumina-treated ZnO dispersed in the ethylhexyl methoxycinnamate/PVP-hexadecene copolymer/methicone mixture);

those marketed under the trademark “Fuji ZnO-SMS-10” by Fuji Pigment (ZnO coated with silica and polymethylsilsesquioxane);

those marketed under the trademark “Nanox Gel TN” by Elementis (ZnO dispersed at 55% in C₁₂-C₁₅ alkyl benzoate with hydroxystearic acid polycondensate).

The uncoated cerium oxide pigments are marketed, for example, under the trademark “Colloidal Cerium Oxide” by Rhone-Poulenc.

The uncoated iron oxide pigments are, for example, marketed by Arnaud under the trademarks “Nanogard WCD 2002 (FE 45B)”, “Nanogard Iron FE 45 BL AQ”, “Nanogard FE 45R AQ” or “Nanogard WCD 2006 (FE 45R)”, or by Mitsubishi under the trademark “TY-220”.

The coated iron oxide pigments are, for example, marketed by Arnaud under the trademarks “Nanogard WCD 2008 (FE 45B FN)”, “Nanogard WCD 2009 (FE 45B 556)”, “Nanogard FE 45 BL 345” or “Nanogard FE 45 BL” or by BASF under the trademark “Oxyde de fer transparent”.

Mixtures of metal oxides are also exemplary, in particular of titanium dioxide and of cerium dioxide, including the mixture of equal weights of titanium dioxide coated with silica and of cerium dioxide coated with silica marketed by Ikeda under the trademark “Sunveil A”, and also the mixture of titanium dioxide and of zinc dioxide coated with alumina, with silica and with silicone, such as the product “M 261” marketed by Kemira, or coated with alumina, with silica and with glycerol, such as the product “M 211” marketed by Kemira.

The additional photoprotective agents are generally present in the compositions according to the invention in proportions ranging from 0.01% to 20% by weight, with respect to the total weight of the composition, and preferably ranging from 0.1% to 10% by weight, with respect to the total weight of the composition.

The compositions according to the invention can also comprise agents for the artificial tanning and/or browning of the skin (self-tanning agents) and more particularly dihydroxyacetone (DHA). They are preferably present in amounts ranging from 0.1% to 10% by weight, with respect to the total weight of the composition.

The aqueous compositions in accordance with the present invention can additionally comprise conventional cosmetic adjuvants selected in particular from among fatty substances, organic solvents, ionic or nonionic and hydrophilic or lipophilic thickeners, softening agents, humectants, opacifiers, stabilizing agents, emollients, silicones, anti-foaming agents, fragrances, preservatives, anionic, cationic, nonionic, zwitterionic or amphoteric surfactants, active principles, fillers, polymers, propellants, basifying or acidifying agents or any other ingredient commonly used in the cosmetics and/or dermatological field.

The fatty substances can be an oil or a wax, other than the non-polar waxes as defined above, or their mixtures. The term “oil” means a compound which is liquid at ambient temperature. The term “wax” means a compound which is solid or substantially solid at ambient temperature and which has a melting point generally of greater than 35° C.

Exemplary oils are mineral oils (liquid paraffin); vegetable oils (sweet almond, macadamia, blackcurrant seed or jojoba oil); synthetic oils, such as perhydrosqualene, fatty alcohols, fatty amides (such as isopropyl lauroyl sarcosinate, marketed under the trademark of “Eldew SL-205” by Ajinomoto), fatty acids or esters (such as C₁₂-C₁₅ alkyl benzoate, marketed under the trademark “Finsolv TN” or “Witconol TN” by Witco, octyl palmitate, isopropyl lanolate, triglycerides, including those of capric/caprylic acids, or dicaprylyl carbonate, marketed under the trademark “Cetiol CC” by Cognis), or oxyethylenated or oxypropylenated fatty esters and ethers; silicone oils (cyclomethicone, polydimethylsiloxanes or PDMSs); fluorinated oils; or polyalkylenes.

Exemplary waxy compounds are carnauba wax, beeswax, hydrogenated castor oil, polyethylene waxes and polymethylene waxes, such as that marketed under the trademark Cirebelle 303 by Sasol.

Exemplary organic solvents are lower alcohols and polyols. The latter can be selected from glycols and glycol ethers, such as ethylene glycol, propylene glycol, butylene glycol, dipropylene glycol or diethylene glycol.

Exemplary hydrophilic thickeners are carboxyvinyl polymers, such as the Carbopols (Carbomers) and the Pemulens (acrylate/C₁₀-C₃₀-alkyl acrylate copolymer); polyacrylamides, such as, for example, the crosslinked copolymers marketed under the trademarks Sepigel 305 (CTFA name: polyacrylamide/C13-14 isoparaffin/Laureth 7) or Simulgel 600 (CTFA name: acrylamide/sodium acryloyldimethyltaurate copolymer/isohexadecane/polysorbate 80) by Seppic; optionally crosslinked and/or neutralized polymers and copolymers of 2-acrylamido-2-methylpropanesulfonic acid, such as the poly(2-acrylamido-2-methylpropanesulfonic acid) marketed by Hoechst under the trademark “Hostacerin AMPS” (CTFA name: ammonium polyacryloyldimethyl taurate) or Simulgel 800, marketed by Seppic (CTFA name: sodium polyacryloyldimethyl taurate/polysorbate 80/sorbitan oleate); copolymers of 2-acrylamido-2-methylpropanesulfonic acid and of hydroxyethyl acrylate, such as Simulgel NS and Sepinov EMT 10, marketed by Seppic; cellulose derivatives, such as hydroxyethylcellulose; polysaccharides and in particular gums, such as xanthan gum; and their mixtures.

Exemplary lipophilic thickeners are synthetic polymers, such as the poly(C₁₀-C₃₀ alkyl acrylates) marketed under the trademarks “Intelimer IPA 13-1” and “Intelimer IPA 13-6” by Landec, or of modified clays, such as hectorite and its derivatives, such as the products marketed under the Bentone names.

Among the active principles, exemplary are:

vitamins (A, C, E, K, PP, and the like) and their derivatives or precursors, alone or as mixtures;

agents for combating pollution and/or agents for combating free radicals;

depigmenting agents and/or propigmenting agents;

anti-glycation agents;

soothing agents;

NO-synthase inhibitors;

agents which stimulate the synthesis of dermal or epidermal macromolecules and/or which prevent their decomposition;

agents which stimulate the proliferation of fibroblasts;

agents which stimulate the proliferation of keratinocytes;

muscle-relaxing agents;

tightening agents;

matifying agents;

keratolytic agents;

desquamating agents;

moisturizing agents;

anti-inflammatory agents;

agents which act on the energy metabolism of the cells;

insect repellents;

substance P or substance CRGP antagonists;

agents for combating hair loss and/or for the regrowth of the hair;

anti-wrinkle agents.

Of course, one skilled in the art will take care to select the additional optional compound or compounds mentioned above and/or their amounts such that the advantageous properties intrinsically associated with the compositions in accordance with the invention are not, or not substantially, detrimentally affected by the envisaged addition or additions.

The compositions according to the invention can be prepared according to techniques well known to a person skilled in the art. They can in particular be provided in the form of a simple or complex (O/W, W/O, O/W/O or W/O/W) emulsion, such as a cream or a milk, or of a cream gel; in the form of an aqueous gel; in the form of a lotion. They can optionally be packaged as an aerosol and be provided in the foam or spray form.

Preferably, the compositions according to the invention are provided in the form of an oil-in-water or water-in-oil emulsion.

The emulsions generally comprise at least one emulsifier selected from among amphoteric, anionic, cationic or nonionic emulsifiers, used alone or as a mixture. The emulsifiers are appropriately selected according to the emulsion to be obtained (W/O or O/W emulsion). The emulsions can also comprise other types of stabilizing agents, such as, for example, fillers or gelling or thickening polymers.

Exemplary emulsifying surfactants which can be used for the preparation of the W/O emulsions are, for example, sorbitan, glycerol or sugar alkyl esters or ethers; silicone surfactants, such as dimethicone copolyols, for example the mixture of cyclomethicone and of dimethicone copolyol marketed under the trademark “DC 5225 C” by Dow Corning, and alkyl dimethicone copolyols, such as lauryl methicone copolyol, marketed under the trademark “Dow Corning 5200 Formulation Aid” by Dow Corning, cetyl dimethicone copolyol, such as the product marketed under the trademark Abil EM 90R by Goldschmidt, and the mixture of cetyl dimethicone copolyol, of polyglycerol (4 mol) isostearate and of hexyl laurate marketed under the trademark Abil WE O9 by Goldschmidt. It is also possible to add thereto one or more coemulsifiers which, advantageously, can be selected from the group consisting of polyol alkyl esters.

Exemplary polyol alkyl esters are of polyethylene glycol esters, such as PEG-30 dipolyhydroxystearate, such as the product marketed under the trademark Arlacel P135 by ICI.

Exemplary esters of glycerol and/or of sorbitan, for example, are polyglycerol isostearate, such as the product marketed under the trademark Isolan GI 34 by Goldschmidt; sorbitan isostearate, such as the product marketed under the trademark Arlacel 987 by ICI; glycerol sorbitan isostearate, such as the product marketed under the trademark Arlacel 986 by ICI, and their mixtures.

For the O/W emulsions, exemplary emulsifiers are nonionic emulsifiers, such as oxyalkylenated (more particularly polyoxyethylenated) esters of fatty acids and of glycerol; oxyalkylenated esters of fatty acids and of sorbitan; oxyalkylenated (oxyethylenated and/or oxypropylenated) fatty acid esters, such as the PEG-100 stearate/glyceryl stearate mixture marketed, for example, by ICI under the trademark Arlacel 165; oxyalkylenated (oxyethylenated and/or oxypropylenated) fatty alcohol ethers; sugar esters, such as sucrose stearate; ethers of fatty alcohol and of sugar, in particular alkyl polyglucosides (APG), such as decyl glucoside and lauryl glucoside, for example marketed by Henkel under the respective names Plantaren 2000 and Plantaren 1200, cetearyl glucoside, optionally as a mixture with cetearyl alcohol, for example marketed under the trademark Montanov 68 by Seppic, under the trademark Tegocare CG90 by Goldschmidt and under the trademark Emulgade KE3302 by Henkel, and arachidyl glucoside, for example in the form of the mixture of arachidyl and behenyl alcohols and of arachidyl glucoside marketed under the trademark Montanov 202 by Seppic. According to a specific embodiment of the invention, the mixture of the alkyl polyglucoside as defined above with the corresponding fatty alcohol can be in the form of a self-emulsifying composition, for example as disclosed in WO-A-92/06778.

Among other emulsion stabilizers, exemplary are polymers of isophthalic acid or sulfoisophthalic acid and in particular the phthalate/sulfoisophthalate/glycol copolymers, for example the diethyleneglycol/phthalate/isophthalate/1,4-cyclohexanedimethanol copolymer (INCI name: Polyester-5) marketed under the trademarks “Eastman AQ polymer” (AQ35S, AQ38S, AQ55S, AQ48 Ultra) by Eastman Chemical.

When an emulsion is involved, the aqueous phase of the latter can comprise a nonionic vesicular dispersion prepared according to known methods (Bangham, Standish and Watkins, J. Mol. Biol., 13, 238 (1965), FR-2,315,991 and FR-2,416,008).

The compositions according to the invention have applications in a large number of treatments, whether regime or regimen, in particular cosmetic treatments, of the skin, lips and hair, including the scalp, in particular for protecting and/or caring for the skin, lips and/or hair and/or for making up the skin and/or lips.

The present invention also features the use of the compositions according to the invention as defined above in the manufacture of products for the cosmetic treatment of the skin, lips, nails, hair, eyelashes, eyebrows and/or scalp, in particular of care products, sun protection products and makeup products.

The cosmetic compositions according to the invention can, for example, be used as makeup product.

The cosmetic compositions according to the invention can, for example, be used as care product and/or sun protection product for the face and/or body with a liquid to semi-liquid consistency, such as milks, relatively smooth creams, cream gels or pastes. They can optionally be packaged in an aerosol and be provided in the foam or spray form.

The compositions according to the invention in the form of vaporizable fluid lotions are applied to the skin or hair in the form of fine particles by means of pressurizing devices. The devices in accordance with the invention are well known to one skilled in the art and comprise non-aerosol pumps or “atomizers”, aerosol containers comprising a propellant and aerosol pumps using compressed air as propellant. The latter are disclosed in U.S. Pat. Nos. 4,077,441 and 4,850,517 (forming an integral part of the content of the description).

The compositions packaged as an aerosol in accordance with the invention generally comprise conventional propellants, such as, for example, hydrofluorinated compounds, dichlorodifluoromethane, difluoroethane, dimethyl ether, isobutane, n-butane, propane or trichlorofluoromethane. They are preferably present in amounts ranging from 15% to 50% by weight, with respect to the total weight of the composition.

In order to further illustrate the present invention and the advantages thereof, the following specific examples are given, it being understood that same are intended only as illustrative and in nowise limitative. In said examples to follow, all parts and percentages are given by weight, unless otherwise indicated.

### SYNTHESIS EXAMPLES

### Example 1

### Preparation of 1,3-dimethylbutyl(benzoylamino)acetate

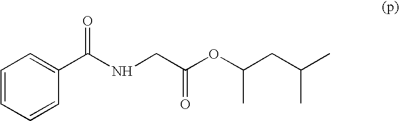

A mixture of hippuric acid (8 g, 0.0446 mol), of 4-methyl-2-pentanol (6.8 ml, 0.0536 mol) and of 98% sulfuric acid (0.41 ml, 0.00446 mol) in 100 ml of toluene is heated at 80° C. for 17 hours. A second addition of 4-methyl-2-pentanol (6.8 ml, 0.0536 mol) and of 98% sulfuric acid (0.41 ml, 0.00446 mol) is carried out and the reaction mixture is brought to reflux for 7 hours. The reaction mixture is evaporated to dryness under reduced pressure. The residue is taken up in ethyl acetate and washed twice with water. The isolated organic phase is dried over sodium sulfate. After filtering and evaporating the solvent, the yellow oil obtained is chromatographed on silica (eluent: heptane/EtOAc 80:20) to give 3 g (22% yield) of clean fractions of the compound of Example 1 in the form of a pale yellow oil.

### Example 2

### Preparation of 2-ethylhexyl(benzoylamino)acetate

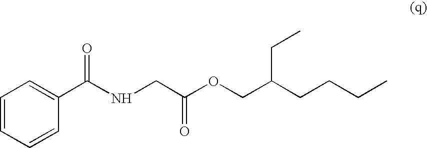

A mixture of hippuric acid (6.25 g, 0.0349 mol), of 2-ethyl-1-hexanol (6 ml, 0.0384 mol) and of 98% sulfuric acid (0.18 ml, 0.00349 mol) in 20 ml of toluene is heated at reflux for 17 hours. The reaction mixture is evaporated to dryness under reduced pressure. The residue is taken up in ethyl acetate and washed twice with water. The isolated organic phase is dried over sodium sulfate. After filtering and evaporating the solvent, the yellow oil obtained is chromatographed on silica (eluent: CH₂Cl₂) to give 8.8 g (87% yield) of clean fractions of the compound of Example 2 in the form of a pale yellow oil.

### Example 3

### Preparation of 2-ethylhexyl[(3-phenylpropanoyl)amino]acetate

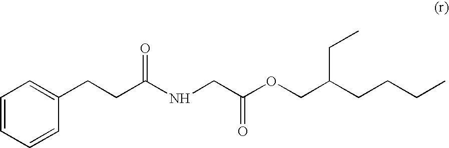

### First stage: Preparation of 2-ethylhexyl aminoacetate

A mixture of glycine (4.36 g, 0.0582 mol), of 2-ethyl-1-hexanol (10 ml, 0.0639 mol) and of 98% sulfuric acid (0.311 ml, 0.00581 mol) in 20 ml of toluene is heated to reflux for 17 hours. 50 ml of toluene are added to the suspension obtained, 0.5 ml of concentrated sulfuric acid is added and the combined mixture is brought to reflux for 24 hours. The reaction mixture is evaporated to dryness under reduced pressure. The residue is chromatographed on silica (eluent: CH₂Cl₂/MeOH 98:2) to give 7 g (87% yield) of clean fractions of 2-ethylhexyl aminoacetate in the form of an orangey oil which is used as is in the following stage.

### Second stage: Preparation of the product of Example 3

3.4 ml of oxalyl chloride (0.0384 mol) are added at 0° C. in 15 minutes to 3-phenylpropanoyl acid (4.81 g, 0.032 mol) dissolved in a mixture of 20 ml of anhydrous acetonitrile and 1 ml of anhydrous DMF. The mixture is left stirring at laboratory temperature for 3 hours. The solvents are evaporated under reduced pressure and the yellow oil obtained is taken up in 20 ml of acetonitrile. Cooling is carried out to −5° C. and the dropwise introduction is carried out of the product from the preceding stage (6 g, 0.032 mol) dissolved in 10 ml of acetonitrile, followed by 16 ml of diisopropylethylamine. The reaction mixture is stirred for 17 hours. It is evaporated to dryness under reduced pressure. The residue is taken up in ethyl acetate and washed with a saturated sodium chloride solution and twice with water. The isolated organic phase is dried over sodium sulfate. After filtering and evaporating the solvent, the orangey oil obtained is chromatographed on silica (eluent: heptane/EtOAc 90:10) to give 3.25 g (31% yield) of clean fractions of the compound of Example 3 in the form of a pale yellow oil.

### Example 4

### Preparation of N-(2-ethylhexyl)-3-phenyl-N-(2-phenylethyl)propanamide

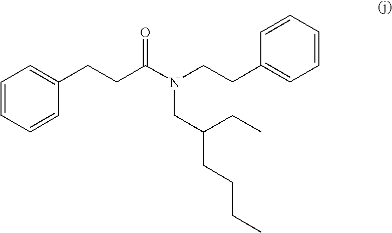

### First stage: Preparation of N-(2-ethylhexyl)-N-(2-phenylethyl)amine

2-Ethyl-1-hexylamine (4 g, 0.0309 mol), dissolved in 16 ml of anhydrous THF, is added in 15 minutes at 0° C. to a suspension of sodium hydride (at 50% in oil, 1.49 g) in 10 ml of anhydrous THF and then the mixture is left for 1 hour at laboratory temperature. 2-Bromoethylbenzene (5.1 ml, 0.037 mol), dissolved in 60 ml of anhydrous THF, is subsequently added in 15 minutes at laboratory temperature. The reaction mixture is left at laboratory temperature for 19 hours and then at 80° C. for 24 hours. After cooling, 100 ml of water are carefully added and extraction is carried out with 3×150 ml of CH₂Cl₂. The organic phase is washed with a saturated NaCl solution, dried over sodium sulfate and then evaporated to dryness under reduced pressure. The yellow oil obtained is chromatographed on silica (eluent: heptane/EtOAc 50:50) to give 3.4 g (47% yield) of clean fractions of N-(2-ethylhexyl)-N-(2-phenylethyl)amine in the form of a yellow oil which is used as in the following stage.

### Second stage: Preparation of the product of Example 4

1.5 ml of oxalyl chloride (0.0145 mol) are added at 0° C. in 10 minutes to dihydrocinnamic acid (2.2 g, 0.0145 mol) dissolved in a mixture of 10 ml of anhydrous acetonitrile and 0.5 ml of anhydrous DMF. The mixture is left stirring at laboratory temperature for 3 hours. The solvents are evaporated under reduced pressure and the orangey oil obtained is taken up in 20 ml of acetonitrile. Cooling is carried out to −5° C. and the dropwise introduction is carried out simultaneously of the product from the preceding stage (3.4 g, 0.0145 mol), dissolved in 10 ml of acetonitrile, and of 7.2 ml of diisopropylethylamine. The reaction mixture is stirred for 17 hours. It is evaporated to dryness under reduced pressure. The residue is taken up in ethyl acetate and washed with a saturated sodium chloride solution and twice with water. The isolated organic phase is dried over sodium sulfate. After filtering and evaporating the solvent, the brown oil obtained is chromatographed on silica (eluent: heptane/EtOAc 90:10) to give 3.98 g (73% yield) of clean fractions of the compound of Example 4 in the form of a pale yellow oil.

### Example 5

### Preparation of 2-phenylethyl 3,4-dimethylbenzoate

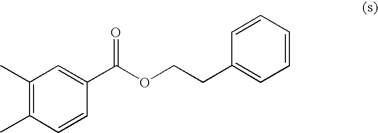

A mixture of 3,4-dimethylbenzoic acid (16.53 g, 0.11 mol), of 2-phenylethanol (15.8 ml, 0.132 mol) and of 98% sulfuric acid (0.6 ml, 0.011 mol) in 170 ml of toluene is heated at 80° C. for 20 hours. The brown reaction mixture is washed with water. After separating the organic phase, the solvent is evaporated under reduced pressure. The residue is taken up in a heptane/AcOEt 97:3 mixture and is filtered through a silica bed. After isolating the clean fractions and after evaporating the solvent, 20.2 g (72% yield) of the compound of Example 5 are obtained in the form of a pale yellow oil.

### Example 6

### Preparation of bis(2-phenylethyl)pyridine-2,4-dicarboxylate

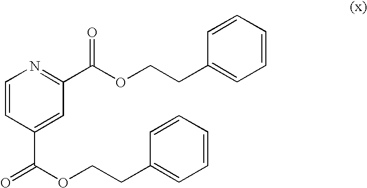

A mixture of 2,4-pyridinedicarboxylic acid (6 g, 0.0359 mol), of 2-phenylethanol (10.16 ml, 0.085 mol) and of 0.6 ml of concentrated sulfuric acid dissolved in 60 ml of toluene is heated at 80° C. for 15 hours. After evaporating the solvents under reduced pressure, the viscous yellow oil obtained is dissolved in dichloromethane and is purified on a silica bed. After evaporating the solvent, 5.8 g (43% yield) of the clean fractions of the product of Example 6 are obtained in the form of a white solid.

### Formulation Examples 7 to 9

The following compositions were prepared and then the photostability of 4-(tert-butyl)-4′-methoxydibenzoylmethane was evaluated for each of them.

| | Ex. 7 | | |
|---|---|---|---|
| Compositions | comparative | Ex. 8 | Ex. 9 |
| Polydimethylsiloxane | 0.5 | 0.5 | 0.5 |
| Preservative | 1 | 1 | 1 |
| Stearic acid | 1.5 | 1.5 | 1.5 |
| Glyceryl monostearate/PEG (100 | 1 | 1 | 1 |
| OE) stearate mixture (Arlacel P165, | | | |
| Uniquema) | | | |
| Deionized water | 73.35 | 73.35 | 73.35 |
| Chelating agent | 0.1 | 0.1 | 0.1 |
| Isopropyl N-lauroylsarcosinate | 10 | 9 | 9 |
| (ELDEW SL-205, Ajinomoto) | | | |
| (amide compound) | | | |
| Mono- or diarylalkyl amide | | 1 | |
| (compound I) | | | |
| Mono or diarylalkyl amide | | | 1 |
| (compound a) | | | |
| 4-(tert-Butyl)-4′- | 2 | 2 | 2 |
| methoxydibenzoylmethane (Parsol | | | |
| 1789, DSM Nutritional Products) | | | |
| Glycerol | 5 | 5 | 5 |
| Xanthan gum | 0.2 | 0.2 | 0.2 |
| Potassium cetyl phosphate | 1 | 1 | 1 |
| (Amphisol K, DSM Nutritional | | | |
| Products) | | | |
| Isohexadecane | 1 | 1 | 1 |
| Acrylic acid/stearyl methacrylate | 0.2 | 0.2 | 0.2 |
| copolymer (Pemulen TR 1, Noveon) | | | |
| Cetyl alcohol | 0.5 | 0.5 | 0.5 |
| Triethanolamine | 0.65 | 0.65 | 0.65 |
| Mixture of cetearyl glucoside and of | 2 | 2 | 2 |
| cetyl/stearyl alcohols (Montanov 68, | | | |
| Seppic) | | | |

Measurement Method:

For each formulation, 3 test samples and 3 control samples were prepared. 2 mg/cm² of formulation are deposited on poly(methyl methacrylate) sheets using a spatula.

The test sheets are exposed for 38 min to a Heraeus Suntest equipped with a xenon lamp having a UV-A flux of 9.5×10⁻³ W/cm² and a UV-B flux of 5.43×10⁴ W/cm². The control sheets are stored for the same time and at the same temperature (38-40° C.) in darkness.

At the end of this time, the screening agents are extracted by immersing each sheet in 50 g of methanol and by subjecting them to ultrasound for 15 min in order to provide for good extraction. The solutions obtained are analyzed by HPLC and UV spectrophotometry.

For each formulation tested, the residual 4-(tert-butyl)-4′-methoxydibenzoylmethane level after exposure is given in the ratio of its optical density (OD) in the exposed sample to its unexposed optical density (OD). The position adopted is the absorption maximum corresponding to butylmethoxydibenzoylmethane: λₘₐₓ=358 nm.

The results obtained are summarized in the following Table 1:

| | Residual % of dibenzoylmethane |
|---|---|
| Compositions | after exposure to UV-A for 1 hour |
| Example 7 | 74% |
| (comparative) | |
| Example 8 | 80% |
| Example 9 | 79% |

An improvement in the photostability of dibenzoylmethane in the presence of an arylalkyl amide compound of formula (I) (compound a) or (II) (compound I) is observed in comparison with isopropyl N-lauroylsarcosinate (Eldew SL-205, Ajinomoto) (amide compound of the prior art).

### Formulation Examples 10 to 12

The following compositions were prepared and then the photostability of 4-(tert-butyl)-4′-methoxydibenzoylmethane was evaluated for each of them according to the method shown in the preceding Examples 7 to 9.

| | Ex. 10 | | |
|---|---|---|---|
| Compositions | (comparative) | Ex. 11 | Ex. 12 |
| Polydimethylsiloxane | 0.5 | 0.5 | 0.5 |
| Preservatives | 1 | 1 | 1 |
| Stearic acid | 1.5 | 1.5 | 1.5 |
| Glyceryl monostearate/PEG (100 | 1 | 1 | 1 |
| OE) stearate mixture (Arlacel | | | |
| P165, Uniquema) | | | |
| Deionized water | 73.35 | 73.35 | 73.35 |
| Sequestering agent | 0.1 | 0.1 | 0.1 |
| 2-Ethylphenyl benzoate (X-Tend | 20 | | |
| 226 ®, ISP) | | | |
| Arylalkyl ester (compound s) | | 20 | |
| Arylalkyl ester (compound v) | | | 20 |
| 4-(tert-Butyl)-4′- | 2 | 2 | 2 |
| methoxydibenzoylmethane | | | |
| (Parsol 1789 ®, DSM Nutritional | | | |
| Products) | | | |
| Glycerol | 5 | 5 | 5 |
| Xanthan gum | 0.2 | 0.2 | 0.2 |
| Potassium cetyl phosphate | 1 | 1 | 1 |
| (Amphisol K, DSM Nutritional | | | |
| Products) | | | |
| Isohexadecane | 1 | 1 | 1 |
| Acrylic acid/stearyl methacrylate | 0.2 | 0.2 | 0.2 |
| copolymer (Pemulen TR 1, | | | |
| Noveon) | | | |
| Cetyl alcohol | 0.5 | 0.5 | 0.5 |
| Triethanolamine | 0.65 | 0.65 | 0.65 |
| Mixture of cetearyl glucoside and | 2 | 2 | 2 |
| of cetyl/stearyl alcohols | | | |
| (Montanov 68, Seppic) | | | |

The results obtained are summarized in the following table:

| | Residual % of dibenzoylmethane |
|---|---|
| Compositions | after exposure to UV-A for 1 hour |
| Example 10 | 52% |
| (comparative) | |
| Example 11 | 74% |
| Example 12 | 65% |

A substantial improvement in the photostability of dibenzoylmethane in the presence of arylalkyl ester (compounds (s) and (v)) is observed in comparison with 2-ethylphenyl benzoate (X-Tend 226®, ISP).

Each patent, patent application, publication, text and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A sunscreen composition comprising at least one dibenzoylmethane UV-A screening agent and an effective photostabilizing amount of at least one arylalkyl amide or ester compound of the following formulae (I), (II) or (III): 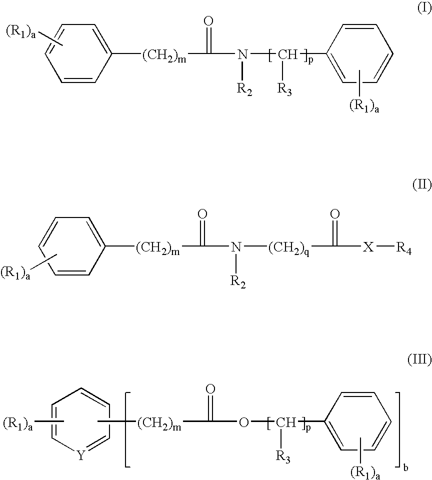
in which:
R₁ is a linear or branched C₁-C₄ alkyl radical,
a is 0-2,
m is 0-4,
b is 1 or 2,
R₂ is H, a linear or branched C₁-C₂₀ alkyl radical or the acetyl group,
R₃ is independently H or methyl,
p is 0-4,
q is 0-3,
X is O, NH or NR₅,
Y is CH or N,
R₄ and R₅ are linear or branched C₁-C₂₀ alkyl radicals,
with the proviso that, in formula (III): if Y=CH, R₃=H, m=0, p=2, then a is other than 0 and R₁ is other than methyl, formulated into a cosmetically acceptable medium therefore.

2. The sunscreen composition as defined by claim 1, comprising a compound of formula (I) selected from the group consisting of the following compounds (a) to (k):

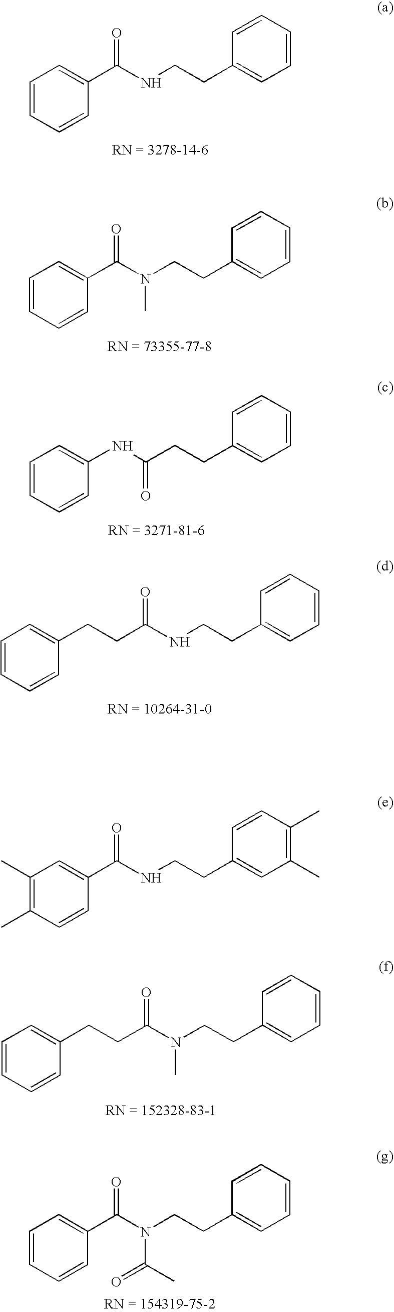

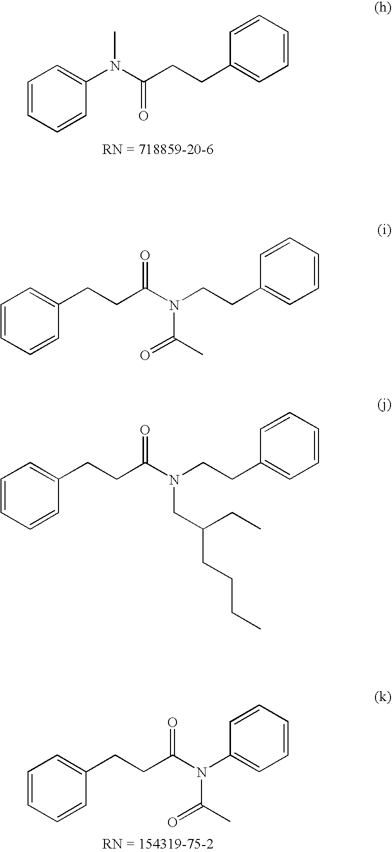

3. The sunscreen composition as defined by claim 1, comprising a compound of formula (II) selected from the group consisting of the following compounds (I) to (r):

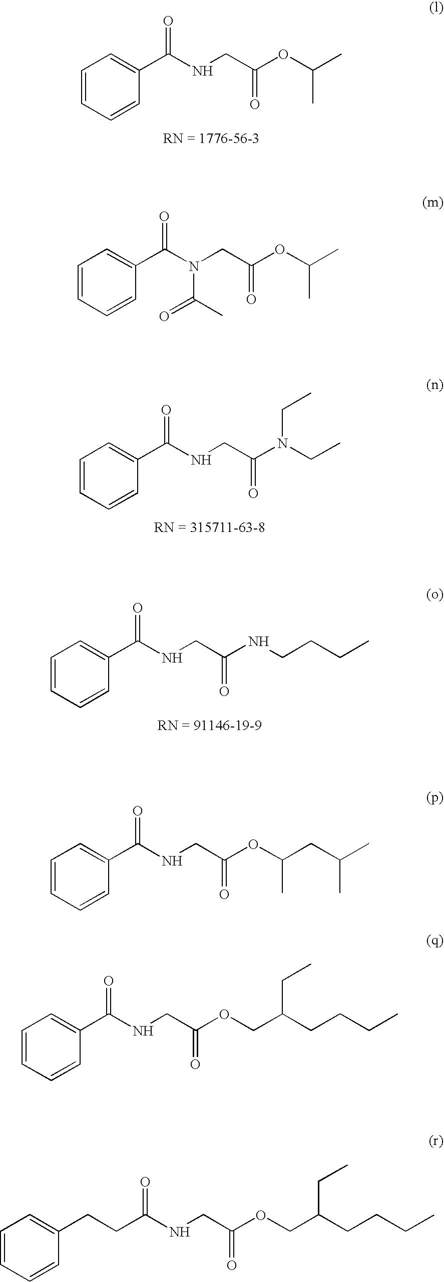

4. The sunscreen composition as defined by claim 1, comprising a compound of formula (III) selected from the group consisting of the following compounds (s) to (x):

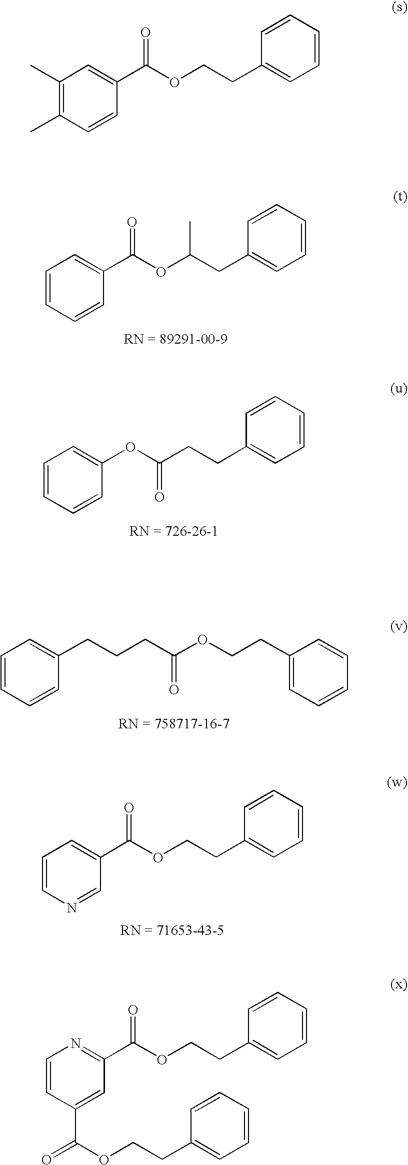

5. The sunscreen composition as defined by claim 1, wherein the arylalkyl amide or ester compound or compounds of formula (I), (II) or (III) are present in proportions ranging from 0.01% to 20% by weight, with respect to the total weight of the composition.

6. The sunscreen composition as defined by claim 1, said at least one dibenzoylmethane screening agent being selected from the group consisting of:
2-methyldibenzoylmethane,
4-methyldibenzoylmethane,
4-isopropyldibenzoylmethane,
4-(tert-butyl)dibenzoylmethane,
2,4-dimethyldibenzoylmethane,
2,5-dimethyldibenzoylmethane,
4,4′-diisopropyldibenzoylmethane,
4,4′-dimethoxydibenzoylmethane,
4-(tert-butyl)-4′-methoxydibenzoylmethane,
2-methyl-5-isopropyl-4′-methoxydibenzoylmethane,
2-methyl-5-(tert-butyl)-4′-methoxydibenzoylmethane,
2,4-dimethyl-4′-methoxydibenzoylmethane,
2,6-dimethyl-4-(tert-butyl)-4′-methoxydibenzoylmethane.

7. The sunscreen composition as defined by claim 5, wherein the at least one dibenzoylmethane screening agent comprises 4-isopropyldibenzoylmethane corresponding to the following formula:

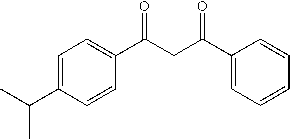

8. The sunscreen composition as defined by claim 5, wherein the at least one dibenzoylmethane screening agent comprises 4-(tert-butyl)-4′-methoxydibenzoylmethane or Butyl Methoxy Dibenzoylmethane of the following formula:

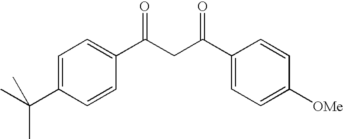

9. The sunscreen composition as defined by claim 1, wherein the at least one dibenzoylmethane screening agent or agents are present at contents which vary from 0.01% to 10% by weight, with respect to the total weight of the composition.

10. The sunscreen composition as defined by claim 1, further comprising other organic or inorganic UV screening agents active in the UV-A and/or UV-B region which are water-soluble or fat-soluble, or insoluble in the common cosmetic solvents.

11. The sunscreen composition as defined by claim 10, further comprising at least one additional organic UV screening agent selected from the group consisting of anthranilates; cinnamic derivatives; salicylic derivatives; camphor derivatives; benzophenone derivatives; β,β-diphenylacrylate derivatives; triazine derivatives; benzotriazole derivatives; benzalmalonate derivatives, benzimidazole derivatives; imidazolines; bis-benzoazolyl derivatives; p-aminobenzoic acid (PABA) derivatives; methylenebis(hydroxyphenylbenzotriazole) derivatives; benzoxazole derivatives; screening polymers and screening silicones; dimers derived from α-alkylstyrene; 4,4-diarylbutadienes; and their mixtures.

12. The sunscreen composition as defined by claim 10, comprising at least one organic UV screening agent or agents selected from the group consisting of:
Ethylhexyl Methoxycinnamate,
Homosalate,
Ethylhexyl Salicylate,
Octocrylene,
Phenylbenzimidazole Sulfonic Acid,
Benzophenone-3,
Benzophenone-4,
Benzophenone-5,
n-Hexyl 2-(4-diethylamino-2-hydroxybenzoyl)benzoate,
4-Methylbenzylidene Camphor, Terephthalylidene Dicamphor Sulfonic Acid,
Disodium Phenyl Dibenzimidazole Tetrasulfonate,
Methylene Bis-Benzotriazolyl Tetramethylbutylphenol,
Ethylhexyl triazone,
Bis-Ethylhexyloxyphenol Methoxyphenyl Triazine,
Diethylhexyl Butamido Triazone,
2,4,6-Tris(dineopentyl 4′-aminobenzalmalonate)-s-triazine,
2,4,6-Tris-(diisobutyl 4′-aminobenzalmalonate)-s-triazine,
2,4,6-Tris(biphenyl-4-yl)-1,3,5-triazine,
2,4,6-Tris(terphenyl)-1,3,5-triazine,
Drometrizole Trisiloxane,
Polysilicone-15,
Dineopentyl 4′-methoxybenzalmalonate,
1,1-Dicarboxy(2,2′-dimethylpropyl)-4,4-diphenylbutadiene,
2,4-Bis[5-1(dimethylpropyl)benzoxazol-2-yl-(4-phenyl)imino]-6-(2-ethylhexyl)imino-1,3,5-triazine,
and mixtures thereof.

13. The sunscreen composition as defined by claim 10, comprising at least one inorganic UV screening agent pigment formed of metal oxides which are or are not treated.

14. The sunscreen composition as defined by claim 13, said at least one pigment being selected from the group consisting of titanium oxide, zinc oxide, iron oxide, zirconium oxide, cerium oxide and mixtures thereof, which are or are not treated.

15. The sunscreen composition as defined by claim 1, further comprising at least one agent for the artificial tanning and/or browning of the skin.

16. The sunscreen composition as defined by claim 1, further comprising at least one adjuvant selected from the group consisting of fatty substances, organic solvents, ionic or nonionic and hydrophilic or lipophilic thickeners, softening agents, humectants, opacifiers, stabilizing agents, emollients, silicones, anti-foaming agents, fragrances, preservatives, anionic, cationic, nonionic, zwitterionic or amphoteric surfactants, active principles, fillers, polymers, propellants, or basifying or acidifying agents.

17. The sunscreen composition as defined by claim 1, in the form of an oil-in-water or water-in-oil emulsion.

18. The sunscreen composition as defined by claim 1, comprising a cosmetic for the treatment of the skin, lips, nails, hair, eyelashes, eyebrows and/or scalp.

19. The sunscreen composition as defined by claim 1, comprising a care product for the skin, lips, nails, hair and/or scalp.

20. The sunscreen composition as defined by claim 1, comprising a makeup.

21. A process for improving the chemical stability with regard to UV radiation of at least one dibenzoylmethane UV-A screening agent wherein said dibenzoylmethane screening agent is combined with at least one arylalkyl amide or ester compound of formula (I), (II) or (III) as defined in claim 1.
